(19) United States
(12) Patent Application Publication
(10) Pub. No.: US 2023/0203437 A1
(43) Pub. Date: Jun. 29, 2023

(54) METHOD FOR PRODUCING NERVE CELL, METHOD FOR PRODUCING MOTOR NEURON, AND METHOD FOR SCREENING NEUROLOGICAL DISEASE THERAPEUTIC DRUG

(71) Applicants: Koji TANABE, Palo Alto, CA (US); I Peace, Inc., Palo Alto, CA (US)

(72) Inventor: Koji TANABE, Palo Alto, CA (US)

(73) Assignees: Koji TANABE, Palo Alto, CA (US); I Peace, Inc., Palo Alto, CA (US)

(21) Appl. No.: 18/146,897

(22) Filed: Dec. 27, 2022

Related U.S. Application Data
(60) Provisional application No. 63/266,082, filed on Dec. 28, 2021.

(51) Int. Cl. C12N 5/0793

(52) U.S. Cl. CPC C12N 5/0619; C12N 2501/60; C12N 2503/02

(57) ABSTRACT

A method for producing nerve cells includes introducing NGN1 as a differentiation factor into stem cells. A method for producing nerve cells includes introducing only NeuroD4 as a differentiation factor into stem cells. A method for producing motor neurons includes introducing NGN2, ISL2, and LHX4 as differentiation factors into stem cells. A method for producing motor neurons includes introducing NGN1, ISL2, and LHX4 as differentiation factors into stem cells. A method for producing motor neurons includes introducing NeuroD4, ISL2, and LHX4 as differentiation factors into stem cells. A method for screening neurological disease therapeutic drugs includes introducing a differentiation factor into pluripotent stem cells derived from neurological disease patients and differentiating the cells into neural cells, applying a candidate drug to cells during differentiation of the pluripotent stem cells into the neural cells or to the differentiated neural cells, and screening the candidate drug on the basis of on the neural cells.

### BACKGROUND

### Field

The present invention relates to a cell technique and relates to a method for producing a nerve cell, a method for producing a motor neuron, and a method for screening a neurological disease therapeutic drug.

### Description of Related Art

Induced pluripotent stem cells (iPS cells) can be transformed into any of the cells that make up the body. Therefore, iPS cells, which can be transformed into various types of somatic cells and tissues, are expected to be used for cell transplantation therapy and drug discovery research. In addition, for example, in 2014, retinal cells produced from iPS cells were applied in transplantation therapy. Not only in Japan, but also in other countries throughout the world, projects are underway to produce brain cells and various organ cells from iPS cells and use them for transplantation therapy.

### CITATION LIST

### Patent Document

Patent Document 1: U.S. Pat. No. 9057053
Patent Document 2: U.S. Pat. No. 9822338
Patent Document 3: U.S. Pat. Application Publication No. 2017/0369840

### Non-Patent Document

Non-Patent Document 1: Di Mao et al., “Chemical decontamination of iPS cell-derived neural cell mixtures,” Chem. Commun ., 2018, 54, 1355-1358

### SUMMARY

An object of the present invention is to provide an efficient method for producing a nerve cell. In addition, an object of the present invention is to provide an efficient method for producing a motor neuron. In addition, an object of the present invention is to provide an efficient method for screening a neurological disease therapeutic drug.

A method for producing a nerve cell according to an embodiment of the present invention includes introducing NGN1 or NeuroD4 as a differentiation factor into a stem cell.

The method for producing the nerve cell according to the embodiment of the present invention may further include introducing MYT1L as a differentiation factor into the stem cell.

In the method for producing the nerve cell according to the embodiment of the present invention, the differentiation factor may be RNA.

In the method for producing the nerve cell according to the embodiment of the present invention, a virus vector may be used when RNA is introduced into stem cells.

In the method for producing the nerve cell according to the embodiment of the present invention, the stem cell may be a pluripotent stem cell.

A method for producing a motor neuron according to an embodiment of the present invention includes introducing NGN2, ISL2, and LHX4 as differentiation factors into a stem cell.

In the method for producing the motor neuron according to the embodiment of the present invention, the NGN2, ISL2, and LHX4 RNAs may be introduced into the stem cell.

In the method for producing the motor neuron according to the embodiment of the present invention, a virus vector may be used when RNA is introduced into the stem cell.

In the method for producing the motor neuron according to the embodiment of the present invention, the stem cell may be a pluripotent stem cell.

The method for producing the motor neuron according to the embodiment of the present invention includes introducing NGN1, ISL2, and LHX4 as differentiation factors into the stem cell.

In the method for producing the motor neuron according to the embodiment of the present invention, the NGN1, ISL2, and LHX4 RNAs may be introduced into the stem cell.

In the method for producing the motor neuron according to the embodiment of the present invention, a virus vector may be used when RNA is introduced into the stem cell.

In the method for producing the motor neuron according to the embodiment of the present invention, the stem cell may be a pluripotent stem cell.

The method for producing the motor neuron according to the embodiment of the present invention includes introducing NeuroD4, ISL2, and LHX4 as differentiation factors into the stem cell.

In the method for producing the motor neuron according to the embodiment of the present invention, the NeuroD4, ISL2, and LHX4 RNAs may be introduced into the stem cell.

In the method for producing the motor neuron according to the embodiment of the present invention, a virus vector may be used when RNA is introduced into the stem cell.

In the method for producing the motor neuron according to the embodiment of the present invention, the stem cell may be a pluripotent stem cell.

A method for screening a neurological disease therapeutic drug according to an embodiment of the present invention includes introducing a differentiation factor into a pluripotent stem cell derived from a neurological disease patient and differentiating the cell into a neural cell, applying a candidate drug to a cell during differentiation of the pluripotent stem cell into the neural cell or to the differentiated neural cell, and screening the candidate drug on the basis of the neural cell.

In the method for screening the neurological disease therapeutic drug according to the embodiment of the present invention, the candidate drug may be screened based on differentiation efficiency of the neural cell.

In the method for screening the neurological disease therapeutic drug according to the embodiment of the present invention, the candidate drug may be screened based on the survival rate of the neural cell or the number of the neural cell.

In the method for screening the neurological disease therapeutic drug according to the embodiment of the present invention, the candidate drug may be screened based on the length of an axon of the neural cell.

In the method for screening the neurological disease therapeutic drug according to the embodiment of the present invention, the candidate drug may be screened based on an action potential of the neural cell.

In the method for screening the neurological disease therapeutic drug according to the embodiment of the present invention, the candidate drug may be screened based on action potential transmission between the neural cells.

In the method for screening the neurological disease therapeutic drug according to the embodiment of the present invention, the candidate drug may be screened based on an expression of a protein in the neural cell.

In the method for screening the neurological disease therapeutic drug according to the embodiment of the present invention, the candidate drug may be screened based on a change in localization of a protein in the neural cell.

In the method for screening the neurological disease therapeutic drug according to the embodiment of the present invention, the candidate drug may be screened based on phosphorylation of a protein in the neural cell.

In the method for screening the neurological disease therapeutic drug according to the embodiment of the present invention, the protein may be a nuclear localized protein.

In the method for screening the neurological disease therapeutic drug according to the embodiment of the present invention, the protein may be a DNA-binding protein.

In the method for screening the neurological disease therapeutic drug according to the embodiment of the present invention, the protein may be a phosphorylated protein.

In the method for screening the neurological disease therapeutic drug according to the embodiment of the present invention, the protein may be at least one selected from the group consisting of TDP-43, phosphorylated TDP-43, tau protein, and a phosphorylated protein.

In the method for screening the neurological disease therapeutic drug according to the embodiment of the present invention, the neurological disease may be amyotrophic lateral sclerosis, Huntington’s disease, or Alzheimer’s disease.

In the method for screening the neurological disease therapeutic drug according to the embodiment of the present invention, the neural cell may be a motor neuron.

The method for screening the neurological disease therapeutic drug according to the embodiment of the present invention may further include culturing the differentiated neural cell on a glial cell or an astrocyte.

The method for screening the neurological disease therapeutic drug according to the embodiment of the present invention may further include removing an undifferentiated cell.

In the method for screening the neurological disease therapeutic drug according to the embodiment of the present invention, the undifferentiated cell may be removed with an anti-cancer agent.

According to the present invention, it is possible to provide an efficient method for producing a nerve cell. In addition, according to the present invention, it is possible to provide an efficient method for producing a motor neuron. In addition, according to the present invention, it is possible to provide an efficient method for screening a neurological disease therapeutic drug.

### BRIEF DESCRIPTION OF DRAWINGS

FIGS. 1A and 1B are images of nerve cells according to Example 1;

FIG. 2 is a graph showing the number of nerve cells according to Example 1;

FIG. 3 is an image of nerve cells according to Example 1;

FIGS. 4A and 4B are images of nerve cells according to Reference Example 1;

FIG. 5 is a graph showing the number of nerve cells according to Example 2;

FIG. 6 is an image of nerve cells according to Example 2;

FIG. 7 is an image of nerve cells according to Example 2;

FIG. 8 is a graph showing the number of nerve cells according to Example 3;

FIGS. 9A and 9B are images of nerve cells according to Example 3;

FIG. 10 is an image of nerve cells according to Example 4;

FIG. 11 is a graph showing the number of nerve cells according to Example 4;

FIG. 12 is a graph showing the number of nerve cells according to Example 5;

FIG. 13 is an image of nerve cells according to Example 5;

FIG. 14 is a graph showing the number of nerve cells according to Example 6;

FIGS. 15A and 15B show an image of nerve cells according to Example 6 and a graph showing the number of MAP2-positive cells;

FIG. 16 is a graph showing the number of MAP2-positive cells according to Example 6;

FIG. 17 is a graph showing the number of TUJ1-positive cells according to Example 6;

FIG. 18 shows images of nerve cells according to Example 7;

FIGS. 19A to 19D show graphs of action potentials of nerve cells according to Example 8;

FIG. 20 is an image showing Western blotting results according to Example 9;

FIG. 21 shows images of nerve cells according to Example 9; and

FIGS. 22A to 22C show images of Western blotting results according to Example 10.

### DETAILED DESCRIPTION

Hereinafter, embodiments of the present invention will be described in detail. Here, the following embodiments exemplify a device and a method for embodying the technical ideas of the invention. The technical ideas of the invention do not specify combinations of constituting members and the like as the following. The technical ideas of the invention can be variously modified within the scope of the claims.

### Method for Producing Nerve Cells and Motor Neurons According to Embodiment

A method for producing nerve cells according to an embodiment includes introducing Neurogenin (NGN) 1 as a differentiation factor into stem cells. The differentiation factor may be NGN1 alone. Alternatively, in the method for producing nerve cells according to the embodiment, as a differentiation factor, in addition to NGN1, Myelin Transcription Factor 1-Like (MYT1L) may be introduced into stem cells. The nerve cells differentiated from the stem cells into which NGN1 is introduced may be induced nerve cells. In addition, the nerve cells differentiated from the stem cells into which NGN1 is introduced may be at least one of cerebral nerve cells, motor nerve cells, excitatory nerve cells, cortical nerve cells, glutamatergic nerve cells, GABAergic nerve cells, and inhibitory nerve cells. The nerve cells differentiated from the stem cells into which NGN1 is introduced may be at least either TUJ1-positive or vGlu-positive. In addition, the method for producing nerve cells according to the embodiment includes introducing only NeuroD4 as a differentiation factor into stem cells. Examples of nerve cells include cortical neurons and excitatory nerve cells. The nerve cells differentiated from the stem cells into which NeuroD4 is introduced may be induced nerve cells. In addition, the nerve cells differentiated from the stem cells into which NeuroD4 is introduced may be at least one of cerebral nerve cells, motor nerve cells, and inhibitory nerve cells. The nerve cells differentiated from the stem cells into which NeuroD4 is introduced may be vGlu-positive.

A method for producing motor neurons according to an embodiment includes introducing NGN2, Islet (ISL)2, and LIM homeobox (LHX)4 as differentiation factors into stem cells. The nerve cells differentiated from the stem cells into which NGN2, ISL2, and LHX4 are introduced may be at least either TUJ1-positive or HB9-positive. In addition, a method for producing motor neurons according to an embodiment includes introducing NGN1, ISL2, and LHX4 as differentiation factors into stem cells. The nerve cells differentiated from the stem cells into which NGN1, ISL2, and LHX4 are introduced may be HB9-positive. In addition, a method for producing motor neurons according to an embodiment includes introducing NeuroD4, ISL2, and LHX4 as differentiation factors into stem cells. The nerve cells differentiated from the stem cells into which NeuroD4, ISL2, and LHX4 are introduced may be HB9-positive.

Here, although the gene symbols are described here as those of humans, this is not intended to limit the species by capital or small letters. For example, denoting in all capital letters does not exclude inclusion of mouse or rat genes . However, in the examples, the gene symbols are shown according to the species actually used.

As the stem cells, pluripotent stem cells, induced pluripotent stem cells (iPS cells) and embryonic stem cells (ES cells) can be used. The stem cells may be human cells or non-human animal cells.

Regarding a culture solution in which the stem cells are cultured, Primate ES Cell Medium, mTeSR1, TeSR2, TeSRE8 (STEMCELL Technologies) or the like may be used.

A medium in which the stem cells are cultured may contain a gel. The gel may contain at least one polymer compound selected from the group consisting of deacylated gellan gum, gellan gum, hyaluronic acid, ramsan gum, diutan gum, xanthan gum, carrageenan, fucoidan, pectin, pectic acid, pectinic acid, heparan sulfate, heparin, heparitin sulfate, keratosulfate, chondroitin sulfate, dermatan sulfate, and rhamnan sulfate, and salts thereof. In addition, the gel medium may contain methyl cellulose. When the gel medium contains methyl cellulose, aggregation between cells is further reduced.

The gel may be a temperature-sensitive gel. The temperature-sensitive gel may be at least one selected from among poly(glycerol monomethacrylate) (PGMA), poly(2-hydroxypropyl methacrylate) (PHPMA), Poly(N-isopropylacrylamide) (PNIPAM), amine terminated, carboxylic acid terminated, maleimide terminated, N-hydroxysuccinimide (NHS) ester terminated, triethoxysilane terminated, Poly(N-isopropylacrylamide-co-acrylamide), Poly(N-isopropylacrylamide-co-acrylic acid), Poly(N-isopropylacrylamide-co-butylacrylate), Poly(N-isopropylacrylamide-co-methacrylic acid), Poly(N-isopropylacrylamide-co-methacrylic acid-co-octadecyl acrylate), and N-Isopropylacrylamide.

The medium in which the stem cells are cultured may contain at least one substance selected from the group consisting of cadherin, laminin, fibronectin, and vitronectin.

In the present disclosure, induction refers to transformation, transdifferentiation (Transdifferentiation or Lineage reprogramming), differentiation induction, cells fate change (Cell fate reprogramming) or the like. The differentiation factor may be RNA. RNA may be mRNA.

Together with the differentiation factor, RNA corresponding to drug-resistant genes may be introduced into the stem cells. Drugs are antibiotics, for example, puromycin, neomycin, blasticidin, G418, hygromycin, and Zeocin. The cells into which RNA corresponding to drug-resistant genes is introduced exhibit drug resistance. For example, differentiation factor RNA and RNA corresponding to drug-resistant genes may be connected with RNA for a 2A peptide such as T2A.

Selection of cells according to drug resistance is desirably performed within 5 days after the differentiation factor is introduced into cells. If cells are selected according to the drug resistance within 5 days, the survival rate of the nerve cells tends to be higher.

The differentiation factor RNA may be modified with at least one selected from the group consisting of pseudouridine (Ψ), 5-methyluridine (5meU), N1-methylpseudouridine (me1Ψ), 5-methoxyuridine (5moU), 5-hydroxymethyluridine (5hmU), 5-formyluridine (5fU), 5-carboxymethyl ester uridine (5camU), thioguanosine (thG), N4-methylcytidine (me4C), 5-methylcytidine (m5C), 5-methoxycytidine (5moC), 5-hydroxymethylcytidine (5hmC), 5-hydroxycytidine (5hoC), 5-formylcytidine (5fC), 5-carboxylcytidine (5caC), N6-methyl-2-aminoadenosine (m6DAP), diaminopurine (DAP), 5-methyluridine (m5U), 2′—O—methyluridine (Um or m2′-OU), 2-thiouridine (s2U), and N6-methyl adenosine (m6A). The differentiation factor RNA may be self-replicating RNA.

The differentiation factor RNA may be polyadenylated. The differentiation factor RNA may be prepared by polyadenylation of RNA transcribed in vitro (IVT) . The differentiation factor RNA may be polyadenylated during IVT using a DNA template that encodes poly(A) ends. The differentiation factor RNA may be capped. In order to maximize efficiency of expression in cells, most RNA molecules may contain a cap.

The differentiation factor RNA may have a 5′cap[m7G(5′)ppp(5′)G] structure. The sequence is a sequence that stabilizes RNA and promotes transcription. 5′triphosphate may be removed from RNA having 5′triphosphate according to a dephosphorylation treatment. RNA may have [3′O—Me—m7G(5′)ppp(5′)G] as Anti-Reverse Cap Analog(ARCA). ARCA is a sequence that is inserted before a transcription start point, doubling the transcription efficiency of RNA. RNA may have a PolyA tail.

For example, the differentiation factor is introduced into the stem cells by a transfection method such as a lipofection method. The lipofection method is a method in which a complex of a nucleic acid, which is a negatively charged substance, and a positively charged lipid is formed through an electrical interaction, and the complex is taken into cells through endocytosis or membrane fusion. The lipofection method has advantages such as less damage to cells, excellent introduction efficiency, simple operation, and less time.

For example, for transfection of differentiation factor RNA, as a transfection reagent, Lipofectamine MessengerMAX (registered trademark) is used. In addition, as the lipofection reagent, Lipofectamine (registered trademark) RNAiMAX (Thermo Fisher Scientific), Lipofectamin (registered trademark) 2000, Lipofectamin (registered trademark) 3000, NeonTransfection System (Thermo Fisher scientific), Stemfect RNA transfection reagent (Stemfect), mRNA—In (registered trademark, Molecular Transfer, Inc.) NextFect (registered trademark) RNA Transfection Reagent (BiooSientific), Amaxa (registered product) Human T cell Nucleofector (registered product) kit (commercially available from Lonza, VAPA-1002), Amaxa (registered product) Human CD34 cell Nucleofector (registered product) kit (commercially available from Lonza, VAPA-1003), and ReproRNA (registered trademark) transfection reagent (STEMCELL Technologies) or the like may be used.

Alternatively, a vector may be used when the differentiation factor RNA is introduced into the stem cells. The vector may be a virus vector. The virus vector may be an RNA virus vector. The RNA virus vector may be a Sendai virus (SeV) vector. The Sendai virus vector has RNA genomes that express RNA for synthesizing differentiation factors that enable stem cells to be differentiated into nerve cells. Alternatively, as the vector, DNA plasmids such as lentivirus, retrovirus, and episomal plasmids may be used.

Sendai viruses that are suspended in a medium are introduced into the stem cells. Sendai viruses recognize cell surface antigens and infect the stem cells.

The density of the stem cells during infection with Sendai viruses is, for example, 0.2×10⁵ cells/well to 1.0×10⁶ cells/well, 0.5×10⁵ cells/well to 8.0×10⁵ cells/well, or 1.0×10⁵ cells/well to 4×10⁵ cells/well in wells of a 12-well plate.

When the differentiation factor RNA is introduced into the stem cells, the amount of RNA is, for example, 100 ng or more and 1000 ng or less, 200 ng or more and 900 ng or less, or 300 ng or more and 800 ng or less.

The titer of Sendai viruses used is, for example, 1×10¹² CIU/mL to 1×10⁵ CIU/mL, 1×10¹⁰ CIU/mL to 1×10⁶ CIU/mL, or 1×10⁹ CIU/mL to 1×10⁷ CIU/mL. The multiplicity of infection (MOI) of Sendai viruses is, for example, 0.1 to 100.0, 1.0 to 50.0, or 1.0 to 20.0 at a time.

Transfection with the differentiation factors may be performed a plurality of times.

The medium used for the transfection with the differentiation factors is, for example, a serum-free or low serum medium such as Plurito Reprogramming Medium (STEMGENT) and Opti-MEM (registered trademark, Gibco). Other examples of mediums used for the transfection with the differentiation factors include stem cell mediums such as mTeSR1 and TeSR2, (registered trademark, STEMCELL Technologies Inc.), and Stem Fit (commercially available from Reprocell Inc.). The mediums used before, during, and after the transfection with the differentiation factors may contain B18R proteins. The B18R proteins alleviate the innate antiviral response of cells. The B18R proteins may be used to inhibit cell death according to an immune response associated with insertion of RNA into cells. However, the medium may be free of B18R proteins or the medium may contain B18R proteins in a dilute concentration such as 0.01% to 1%.

After the transfection with the differentiation factors or during the multiple transfections with the differentiation factors, the medium is replaced with a medium suitable for neural cells (hereinafter referred to as a “neural differentiation medium”). Examples of neural differentiation mediums include NDiff 227 (registered trademark, TAKARA BIO INC.), Neurobasal (registered trademark, Thermo Fisher Scientific Inc.), Human ES/iPS Neuronal Differentiation Medium (Merck KGaA) , and N2B27 medium. The N2B27 medium is prepared, for example, by adding 10 mL of B27 and 5 mL of N2 supplement (Thermo Fisher Scientific Inc.) to 500 mL of DMEMF12.

When RNA corresponding to the drug-resistant genes is introduced into the stem cells together with the differentiation factors, cells that exhibit drug resistance may be selected during or after the transfection. For example, when RNA corresponding to puromycin-resistant genes is introduced into the stem cells, if cells after transfection are exposed to puromycin, it is possible to kill cells other than the cells into which the differentiation factors are introduced and select the cells into the which differentiation factors are introduced.

Differentiated nerve cells may be cultured on glial cells or astrocytes.

Whether the stem cells are differentiated into cerebral nerve cells such as cortical neurons and excitatory nerve cells is confirmed from whether at least one of TUJ1, vGlu, SATB2, CTIP2, and REELIN is positive. Whether the stem cells are differentiated into motor neurons is confirmed from whether at least one of TUJ1, HB9, chAT, ISL1, and ISL2 is positive.

### Example 1: Production of Nerve Cells

A plate coated with a solubilized basement membrane matrix (Matrigel, Corning) was prepared. In addition, 1×10⁵, 2×10⁵, or 4×10⁵ single iPS cells were suspended in 1 mL of a human ES/iPS cell maintenance medium (mTeSR1, STEMCELL Technologies), the suspension was added to the plate, and the cells were seeded, and left for one day.

A 1.5 mL microcentrifuge tube A and a 1.5 mL microcentrifuge tube B were prepared.

50 µL of a low serum medium (Opti-MEM (registered trademark), Gibco) was put into the tube A, 1.5 µL of an mRNA introduction reagent (Lipofectamine MessengerMax (registered trademark), Invitrogen) was added thereto, and mixed well to prepare a first reaction solution.

50 µL of a low serum medium (Opti-MEM (registered trademark) , Gibco) was put into the tube B, 500 ng of NGN1 mRNA connected to mRNA corresponding to puromycin-resistant genes was added and mixed well to prepare a second reaction solution.

The second reaction solution was added to the first reaction solution in the tube A to prepare a mixed reaction solution, and the tube A was then gently tapped at room temperature for 10 minutes so that liposomes were formed. Next, the mixed reaction solution was added to the plate, and left at 37° C. for 6 to 8 hours. Thereby, the cells were transfected with mRNA (Day0). Next, all the medium was removed from the plate, and 1 mL of a human ES/iPS cell maintenance medium (mTeSR1, STEMCELL Technologies) containing B18R recombinant proteins at a concentration of 200 ng/ mL was put onto the plate, and left at 37° C. overnight.

In the same manner as the previous day, the cells were transfected with mRNA (Day1). Next, all the medium was removed from the plate, 1 mL of a human ES/iPS cell maintenance medium (mTeSR1, STEMCELL Technologies) containing B18R recombinant proteins at a concentration of 200 ng/ mL and puromycin at a concentration of 2 µg/µL was put into the plate and left at 37° C. overnight. The next day, in the same manner as the previous day, the cells were transfected with mRNA (Day2) .

In the same manner as the previous day, the cells were transfected with mRNA (Day3). Next, all the medium was removed from the plate, and 1 mL of a neural differentiation medium (STEMdiff Neural Induction Medium, registered trademark, STEMCELL Technologies) containing B18R recombinant proteins at a concentration of 200 ng/ mL and puromycin at a concentration of 2 µg/µL was put into the plate and left at 37° C. overnight. The next day, in the same manner as the previous day, the cells were transfected with mRNA (Day4).

On the 5^{th} day after introduction of NGN1 into the cells, drug-resistant cells were selected using puromycin.

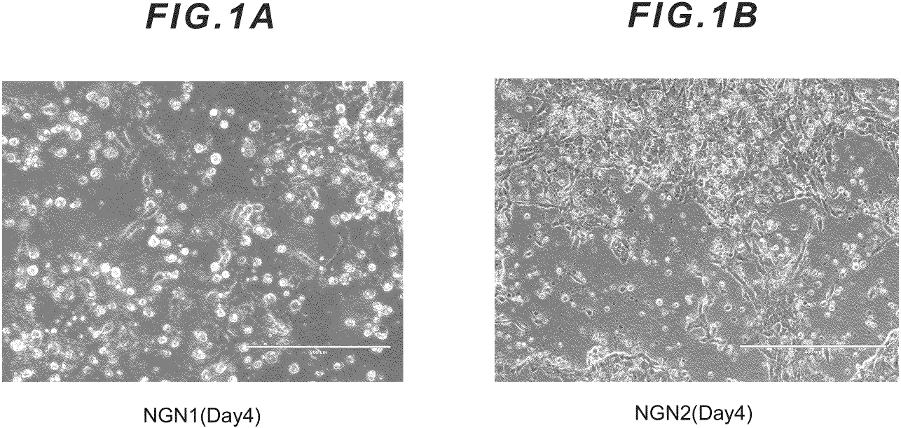

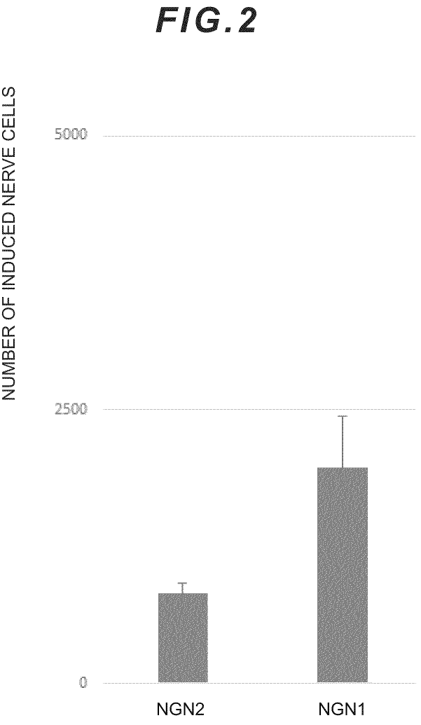

As shown in FIG. 1A, on the 4^{th} day after introduction of NGN1, the stem cells were differentiated into nerve cells. As shown in FIG. 1B, the number of nerve cells differentiated from the cells into which NGN2 was introduced under the same conditions was small. As shown in FIG. 2, on the 14^{th} day after transfection, the number of the nerve cells differentiated with NGN1 was more than double the number of the nerve cells differentiated with NGN2. Therefore, NGN1 showed improved differentiation efficiency than NGN2.

Then, the medium was removed from the plate, and the cells were washed with PBS. Next, 4% PFA was put into the plate and reacted at 4° C. for 15 minutes to fix the cells. In addition, the cells were washed with PBS twice, and primary antibodies were then diluted in a PBS medium containing 5% CCS and 0.1% triton and added to the plate. As the primary antibodies, mouse monoclonal antibodies against TUJ1 and mouse monoclonal antibodies against vGlu, which are markers for nerve cells, were used.

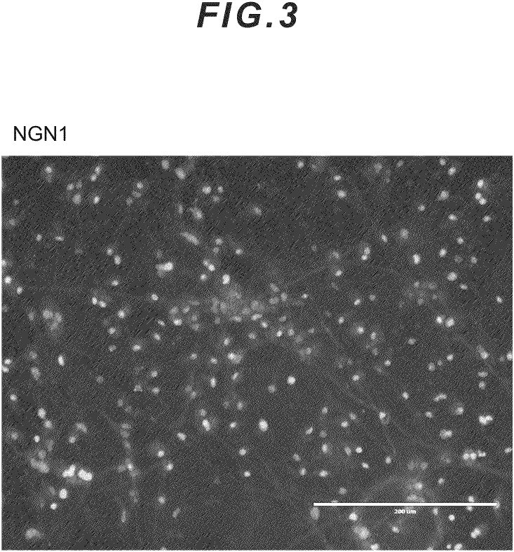

After reacting at room temperature for 1 hour, PBS was added to the plate and mixed well in the plate and the PBS was then discarded. Again, PBS was added and discarded, and a solution containing fluorescently-labeled donkey anti-mouse IgG(H+L) secondary antibodies (Alexa Fluor, registered trademark, 555, Conjugate, Invitorogen)) was added to the plate and reacted at room temperature for 30 minutes . Then, the cells were washed with PBS twice and observed under a fluorescence microscope. As a result, it was confirmed that the cells differentiated from iPS cells expressed TUJ1 which is a marker for nerve cells. In addition, as shown in FIG. 3, it was confirmed that the cells differentiated from iPS cells expressed vGlu which is a marker for nerve cells.

### Reference Example 1

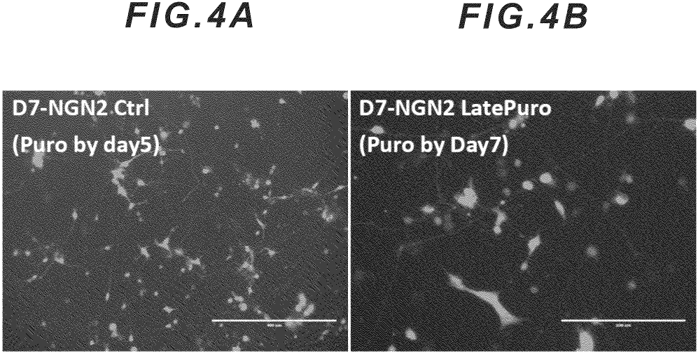

iPS cells were differentiated into nerve cells in the same manner as in Example 1 except that only NGN2 mRNA was used as the differentiation factor. In one group, on the 5^{th} day after introduction of NGN2 into the cells, drug-resistant cells were selected using puromycin, and in another group, on the 7^{th} day after introduction of NGN2 into the cells, drug-resistant cells were selected using puromycin. As a result, as shown in FIGS. 4A and 4B, the nerve cells in which the drug-resistant cells were selected using puromycin on the 5^{th} day had a higher survival rate than the nerve cells in which the drug-resistant cells were selected using puromycin on the 7^{th} day.

### Example 2: Production of Nerve Cells

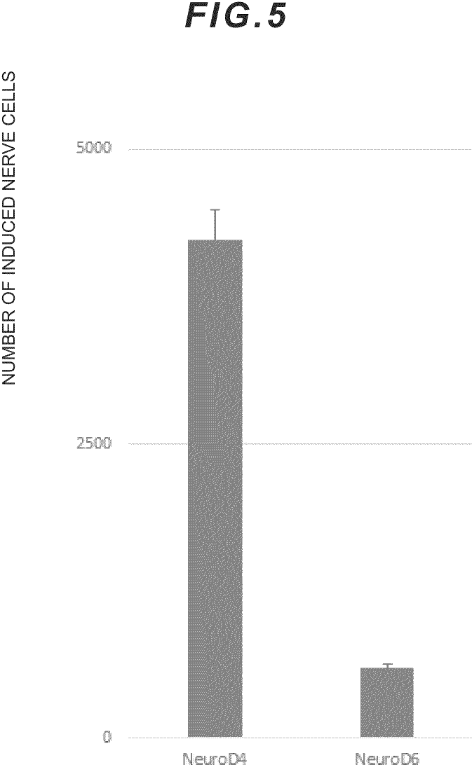

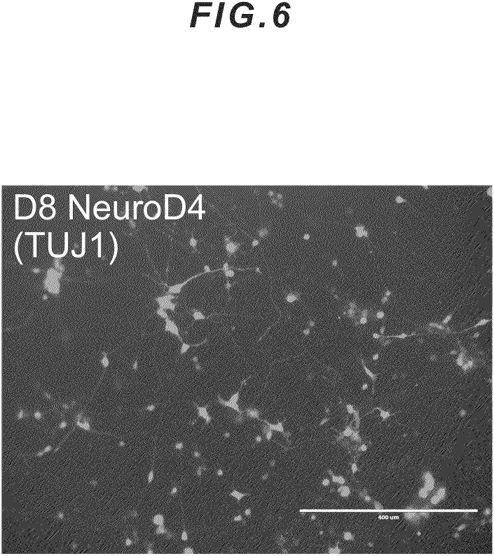

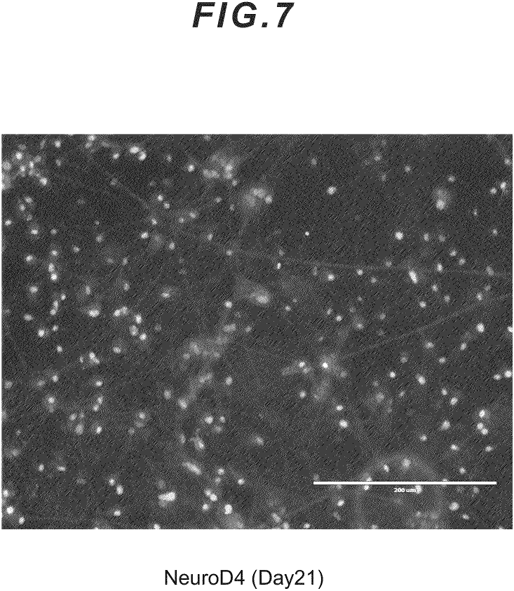

iPS cells were differentiated into nerve cells in the same manner as in Example 1 except that only 500 ng of NeuroD4 mRNA was used as the differentiation factor. As shown in FIG. 5, on the 14^{th} day after transfection, the number of nerve cells differentiated with NeuroD4 was more than double the number of nerve cells differentiated with NeuroD6 under the same conditions. Therefore, NeuroD4 showed improved differentiation efficiency than NeuroD6. In addition, as shown in FIG. 6, it was confirmed on the 8^{th} day that the cells differentiated from iPS cells expressed TUJ1 which is a marker for nerve cells. In addition, as shown in FIG. 7, it was confirmed on the 21^{st} day that the cells differentiated from iPS cells expressed vGlu which is a marker for nerve cells.

### Example 3: Production of Motor Neurons

Differentiation factors were introduced into iPS cells in the same manner as in Example 1 except that, as differentiation factors, 500 ng of NGN2 mRNA, 250 ng of ISL2 mRNA, and 250 ng of LHX4 mRNA were used.

On Day5, the cells were separated from the plate. An N2B27 medium was put onto the plate on which glial cells or astrocytes were cultured in advance, and the cells into which differentiation factors were introduced were seeded on the glial cells or the astrocytes and left at 37° C. overnight. Then, the cells were cultured while replacing the medium every 4 or 5 days.

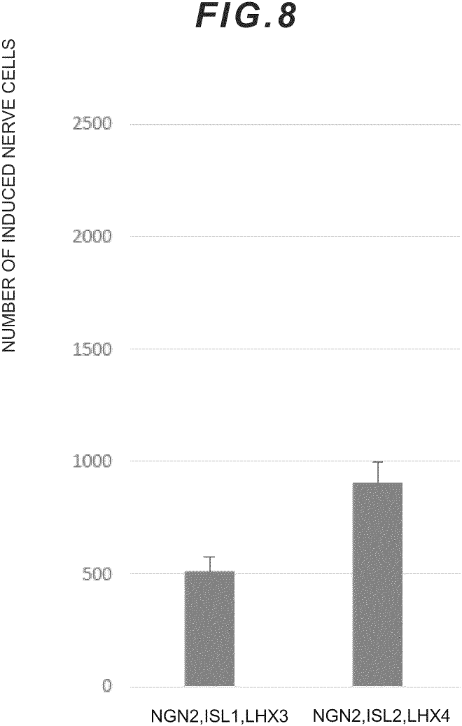

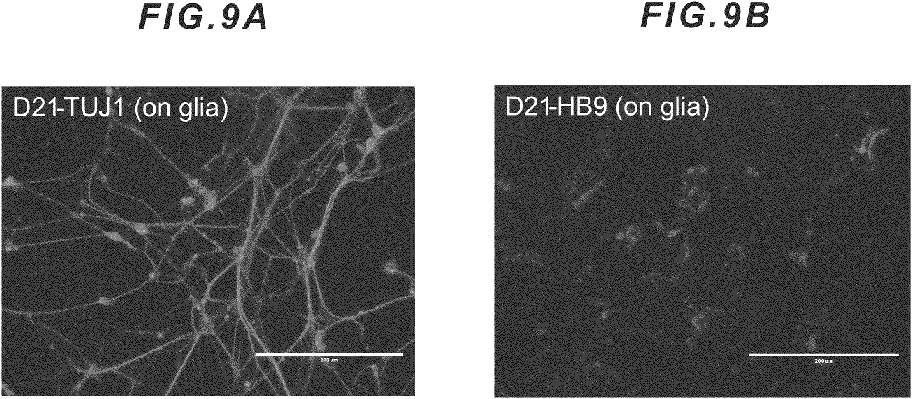

As shown in FIG. 8, on the 14^{th} day after the transfection, the number of nerve cells differentiated with NGN2, ISL2, and LHX4 was larger than the number of nerve cells differentiated with NGN2, ISL1, and LHX3 under the same conditions. Therefore, the combination of NGN2, ISL2, and LHX4 showed improved differentiation efficiency than the combination of NGN2, ISL1, and LHX3. In addition, as shown in FIGS. 9A and 9B, it was confirmed on the 21^{st} day that the cells differentiated from iPS cells expressed TUJ1 and HB9 which are motor neuron markers.

### Example 4: Production of Motor Neurons

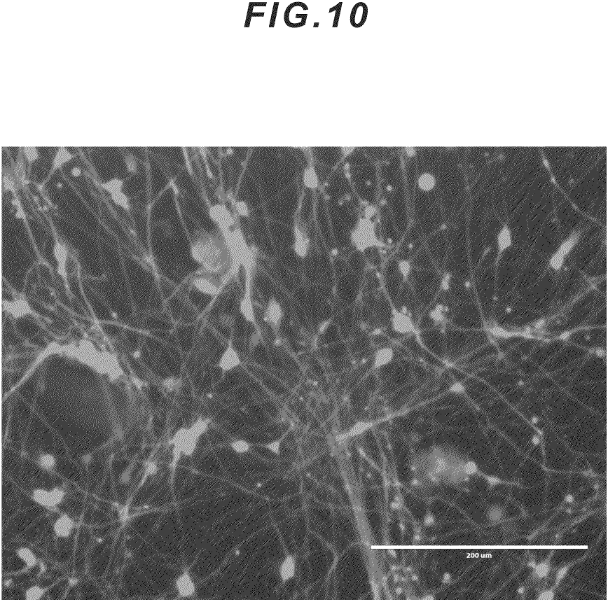

iPS cells were differentiated into nerve cells in the same manner as in Example 3 except that, as differentiation factors, 500 ng of NGN1 mRNA, 250 ng of ISL2 mRNA, and 250 ng of LHX4 mRNA were used. As a result, as shown in FIG. 10, it was confirmed on the 21^{st} day that the cells differentiated from iPS cells expressed HB9 which is a motor neuron marker.

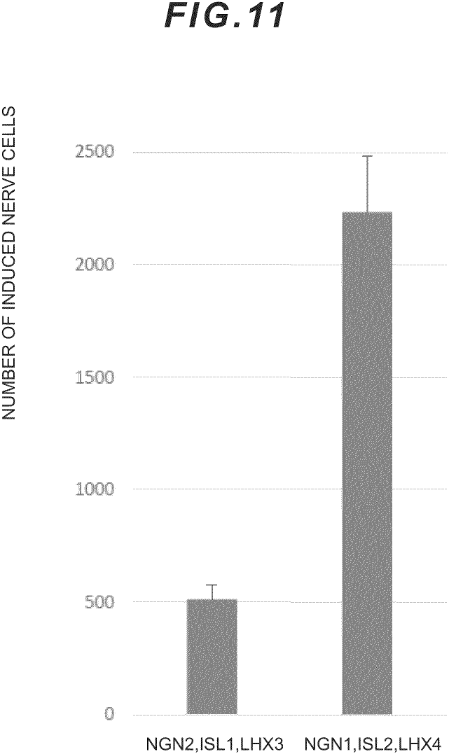

As shown in FIG. 11, on the 14^{th} day after the transfection, the number of the nerve cells differentiated with NGN1, ISL2, and LHX4 was larger than the number of the nerve cells differentiated with NGN2, ISL1, and LHX3 under the same conditions. Therefore, the combination of NGN1, ISL2, and LHX4 showed improved differentiation efficiency than the combination of NGN2, ISL1, and LHX3.

### Example 5: Production of Motor Neurons

iPS cells were differentiated into nerve cells in the same manner as in Example 3 except that, as differentiation factors, 500 ng of NeuroD4 mRNA, 250 ng of ISL2 mRNA, and 250 ng of LHX4 mRNA were used.

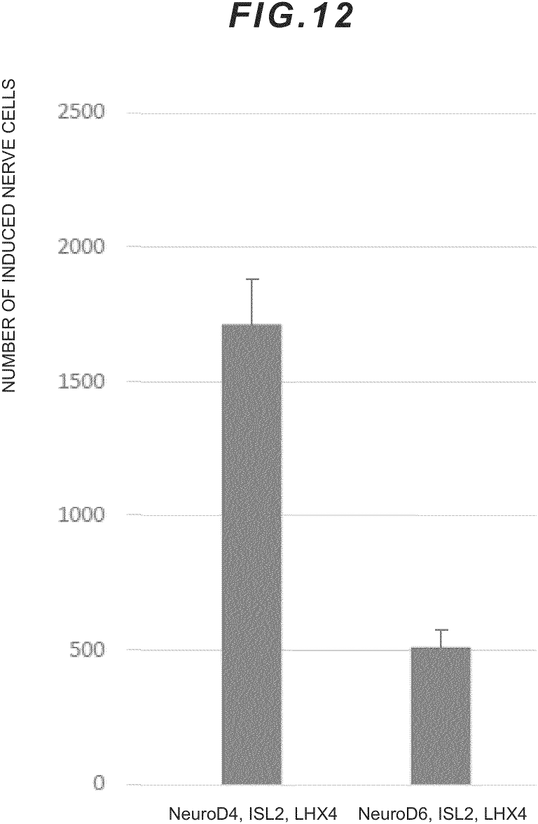

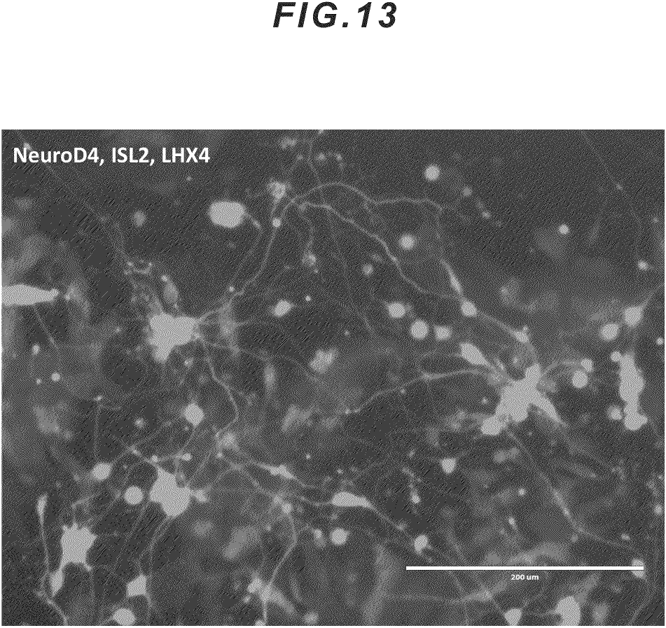

As shown in FIG. 12, on the 14^{th} day after the transfection, the number of the nerve cells differentiated with NeuroD4, ISL2, and LHX4 was larger than the number of the nerve cells differentiated with NeuroD6, ISL2, and LHX4 under the same conditions. Therefore, the combination of NeuroD4, ISL2, and LHX4 showed improved differentiation efficiency than the combination of NeuroD6, ISL2, and LHX4. In addition, as shown in FIG. 13, it was confirmed on the 21^{st} day that the cells differentiated from iPS cells expressed HB9 which is a motor neuron marker.

Method for Screening Neurological Disease Therapeutic Drug According to Embodiment A method for screening a neurological disease therapeutic drug according to an embodiment includes introducing a differentiation factor into pluripotent stem cells derived from a neurological disease patient and differentiating the cells into neural cells, applying a candidate drug to cells during differentiation of the pluripotent stem cells into the neural cells or to the differentiated neural cells, and screening the candidate drug on the basis of the differentiated neural cells. Here, the patients are not limited to humans but include non-human animals . The pluripotent stem cells may be iPS cells .

Examples of the neurological diseases include amyotrophic lateral sclerosis (ALS), Huntington’s disease, and Alzheimer’s disease, but the present invention is not limited thereto. The pluripotent stem cells derived from the neurological disease patients may be produced from somatic cells of the neurological disease patients. Examples of the somatic cells of the neurological disease patients include fibroblasts, blood cells, epithelial cells, somatic stem cells, keratinocytes, dermal papilla cells, and dental pulp stem cells, but the present invention is not limited thereto. iPS cells are induced by introducing reprogramming factors such as OCT¾, KLF4, c-MYC, SOX2, LIN28, and NANOG into the somatic cells derived from the neurological disease patients, but the reprogramming factors are not limited thereto.

The differentiation factors introduced into the stem cells are DNA, RNA, or proteins. RNA may be mRNA. Examples of the differentiation factors include Neurogenin (NGN), Myelin Transcription Factor 1-Like (MYT1), NeuroD, Islet (ISL), LIM homeobox (LHX), Achaete-Scute Homolog (ASCL), and Distal-Less Homeobox (DLX), but the present invention is not particularly limited.

NGN may be NGN1 or NGN2. NGN1 alone may be introduced into stem cells. NeuroD may be NeuroD1, NeuroD2, NeuroD4 or NeuroD6. NeuroD4 alone may be introduced into stem cells. ISL may be ISL1 or ISL2. LHX may be LHX1, LHX2, LHX3, LHX4 or LHX5. ASCL may be ASCL1, ASCL2, ASCL3, ASCL4 or ASCL5. DLX may be DLX1, DLX2 or DLX5.

NGN1, NGN2, NeuroD4, and NeuroD6 alone can differentiate the stem cells into cortical neurons. The combination of NGN2, ISL1, and LHX3 can differentiate the stem cells into motor neurons. The combination of NGN2, ISL2, and LHX4 can differentiate the stem cells into motor neurons. The combination of NGN1, ISL2, and LHX4 can differentiate the stem cells into motor neurons. The combination of NeuroD4, ISL2, and LHX4 can differentiate the stem cells into motor neurons. The combination of NeuroD6, ISL2, and LHX4 can differentiate the stem cells into motor neurons.

NGN2, ASCL1, MYT1L, and DLX2 are switch proteins required for nerve cell generation. NGN2 can differentiate stem cells into excitatory nerve cells. Each of ASCL1, MYT1L, and DLX2 can differentiate the stem cells into inhibitory nerve cells. The combination of ASCL1 and MYT1L can differentiate the stem cells into excitatory nerve cells, inhibitory nerve cells, and dopamine-producing nerve cells. The combination of ASCL1, DLX2, and MYT1L can differentiate the stem cells into inhibitory nerve cells.

The method for differentiating the pluripotent stem cells into the neural cells may be the same as in the above embodiment. The differentiated neural cells may be cultured on glial cells or astrocytes. Undifferentiated cells may be removed. The undifferentiated cells include incompletely differentiated cells. For example, a gene that is specifically expressed in undifferentiated cells may be used to control drug toxicity and allow only differentiated cells to survive. For example, since undifferentiated cells express alkaline phosphatase, only cells expressing alkaline phosphatase may be killed. In addition, undifferentiated cells have a higher proliferative ability than differentiated cells. Therefore, undifferentiated cells may be killed by a drug that specifically kills cells with a high proliferative ability such as an anti-cancer agent. Examples of drugs that kill undifferentiated cells include phosphorylated 7 -Ethyl -10 -hydroxycamptothecin (SN38-P).

The neural cells include nerve cells and glial cells. Examples of the differentiated neural cells include motor neurons, cortical neurons, nerve stem cells, nerve precursor cells, inhibitory nerve cells, excitatory nerve cells, dopamine-producing nerve cells, oligodendrocyte precursor cells, and oligodendrocytes, but the present invention is not limited thereto.

For example, whether the stem cells are differentiated into the neural cells is confirmed from whether at least one selected from among NGN such as MAP2, TUJ1 (β-IIITubulin), BRN2, HOMER1, and NGN2, ASCL such as PSA-NCAM, MUNC13-1, SATB2, vGLUT, chAT, HB9, LHX3, GAD65, GAD67, TH, and ASCL1 (MASH1), vGAT, SOX1, SOX2, CD133, Nestin, HB9, ISL1, 04, PLP1, MOG, and MBP is positive.

NGN2 is a marker for excitatory nerve cells. TUJ1 (P-IIITubulin), MAP2, BRN2, ASCL1 (MASH1) and PSA-NCAM are markers for nerve cells. HOMER1 and MUNC13-1 are markers for mature nerve cells forming synapses. SATB2 is a marker for cerebral cortical nerve cells. vGLUT is a marker for excitatory nerve cells such as glutamatergic nerve cells. chAT is a marker for motor nerve cells such as cholinergic nerve cells. HB9 is a marker for motor nerve cells. GAD65 and GAD67 are markers for inhibitory nerve cells such as GABAergic nerve cells. TH is a marker for dopamine-producing nerve cells. vGAT is a marker for inhibitory nerve cells. SOX1, SOX2, CD133, and Nestin are markers for nerve stem cells. LHX3, HB9 and ISL1 are markers for motor nerve cells. 04, PLP1, MOG, and MBP are markers for oligodendrocyte precursors. GFAP and CD44 are markers for astrocyte precursors and astrocytes.

When the candidate drug is screened, for example, the candidate drug is added to a medium during the differentiation of the stem cells into the neural cells and/or a medium after the differentiation of the stem cells into the neural cells. The amount of the candidate drug added and the culture time after addition are appropriately set based on the purpose.

According to the findings of the inventors, the efficiency with which iPS cells derived from neurological disease patients are differentiated into neural cells with differentiation factors is lower than the efficiency with which iPS cells derived from healthy subjects are differentiated into neural cells with a differentiation factor. Therefore, the positive rate of markers for neural cells differentiated from iPS cells derived from neurological disease patients is lower than the positive rate of markers for neural cells differentiated from iPS cells derived from healthy subjects. In addition, the survival rate of neural cells or the number of neural cells differentiated from iPS cells derived from neurological disease patients is lower than the survival rate of neural cells or the number of neural cells differentiated from iPS cells derived from healthy subjects. Therefore, candidate drugs for neurological diseases may be screened based on the extent to which iPS cells derived from neurological disease patients improve efficiency of differentiating into neural cells. For example, candidate drugs for neurological diseases that increase the positive rate of markers for neural cells differentiated from iPS cells derived from neurological disease patients may be screened. In addition, for example, candidate drugs for neurological diseases that increase the survival rate of neural cells or the number of neural cells differentiated from iPS cells derived from neurological disease patients may be screened. Alternatively, candidate drugs for neurological diseases that inhibit cell death of neural cells differentiated from iPS cells derived from neurological disease patients may be screened.

According to the findings of the inventors, axons of neural cells differentiated from iPS cells derived from neurological disease patients are shorter than axons of neural cells differentiated from iPS cells derived from healthy subjects. Therefore, candidate drugs for neurological diseases may be screened based on the extent to which the length of axons of neural cells differentiated from iPS cells derived from neurological disease patients is lengthened. For example, candidate drugs for neurological diseases that lengthen axons of neural cells differentiated from iPS cells derived from neurological disease patients may be screened.

According to the findings of the inventors, the action potential of neural cells differentiated from iPS cells derived from neurological disease patients is lower than that of the action potential of neural cells of healthy subjects. Therefore, candidate drugs for neurological diseases may be screened based on the extent to which the intensity of the action potential of neural cells differentiated from iPS cells derived from neurological disease patients is improved. For example, candidate drugs for neurological diseases that improve the action potential of neural cells differentiated from iPS cells derived from neurological disease patients may be screened.

According to the findings of the inventors, the coordination of action potential transmission between neural cells differentiated from iPS cells derived from neurological disease patients is weaker than the coordination of action potential transmission between neural cells of healthy subjects. Therefore, candidate drugs for neurological diseases may be screened based on the extent to which the coordination of action potential transmission between neural cells differentiated from iPS cells derived from neurological disease patients is improved. For example, candidate drugs for neurological diseases that increase the coordination of action potential transmission between neural cells differentiated from iPS cells derived from neurological disease patients may be screened.

According to the findings of the inventors, the number of synapses formed in neural cells differentiated from iPS cells derived from neurological disease patients is smaller than the number of synapses formed in neural cells of healthy subjects. Therefore, candidate drugs for neurological diseases may be screened based on the extent to which the number of synapses formed in neural cells differentiated from iPS cells derived from neurological disease patients increases. For example, candidate drugs for neurological diseases that increase the number of synapses formed in neural cells differentiated from iPS cells derived from neurological disease patients may be screened.

According to the findings of the inventors, an expression of proteins in neural cells differentiated from iPS cells derived from neurological disease patients differs from an expression of proteins in neural cells of healthy subjects. Therefore, candidate drugs for neurological diseases may be screened based on the extent to which an expression of proteins in neural cells differentiated from iPS cells derived from neurological disease patients is remedied.

For example, in neural cells of healthy subjects, DNA-binding proteins such as TDP-43 are localized in the nucleus, on the other hand, in neural cells differentiated from iPS cells derived from neurological disease patients, DNA-binding proteins such as TDP-43 are localized in the cytoplasm. Therefore, candidate drugs for neurological diseases may be screened based on the extent to which localization of nuclear localized proteins such as DNA-binding proteins in neural cells differentiated from iPS cells derived from neurological disease patients is remedied. For example, candidate drugs for neurological diseases that localize DNA-binding proteins in the nucleus in neural cells differentiated from iPS cells derived from neurological disease patients may be screened.

In addition, for example, a larger amount of phosphorylated TDP-43 is expressed in neural cells differentiated from iPS cells derived from neurological disease patients than in neural cells of healthy subjects. Therefore, candidate drugs for neurological diseases may be screened based on the extent to which an amount of expression of phosphorylated TDP-43 in neural cells differentiated from iPS cells derived from neurological disease patients decreases. For example, candidate drugs for neurological diseases that decrease an amount of expression of phosphorylated TDP-43 in neural cells differentiated from iPS cells derived from neurological disease patients may be screened.

In addition, for example, a larger amount of Tau proteins and phosphorylated Tau proteins is expressed in neural cells differentiated from iPS cells derived from neurological disease patients than in neural cells of healthy subjects. Therefore, candidate drugs for neurological diseases may be screened based on the extent to which an amount of expression of Tau proteins and phosphorylated Tau proteins in neural cells differentiated from iPS cells derived from neurological disease patients decreases. For example, candidate drugs for neurological diseases that decrease an amount of expression of Tau proteins and phosphorylated Tau proteins in neural cells differentiated from iPS cells derived from neurological disease patients may be screened. Examples of phosphorylated site of Tau protein are Ser199, Ser202, Thr205, Ser214, Thr231, Ser396, Ser404, and Ser416. The phosphorylated sites may be Ser202 and Thr205. The phosphorylated site may be Ser396.

### Example 6: Differentiation Efficiency

iPS cells derived from healthy subjects, iPS cells derived from amyotrophic lateral sclerosis (ALS) patients, iPS cells derived from Huntington’s disease patients, and iPS cells derived from Alzheimer’s disease patients were prepared. iPS cells were differentiated into motor nuerons in the same manner as in Example 1 except that, as the differentiation factor, only 500 ng of NGN2 mRNA was used.

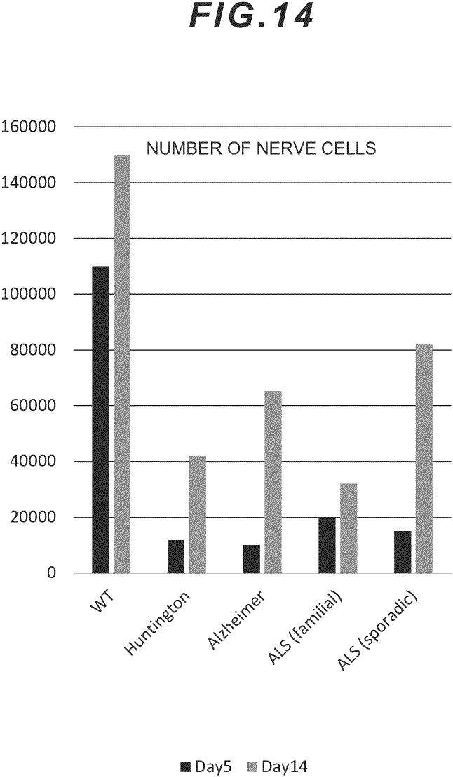

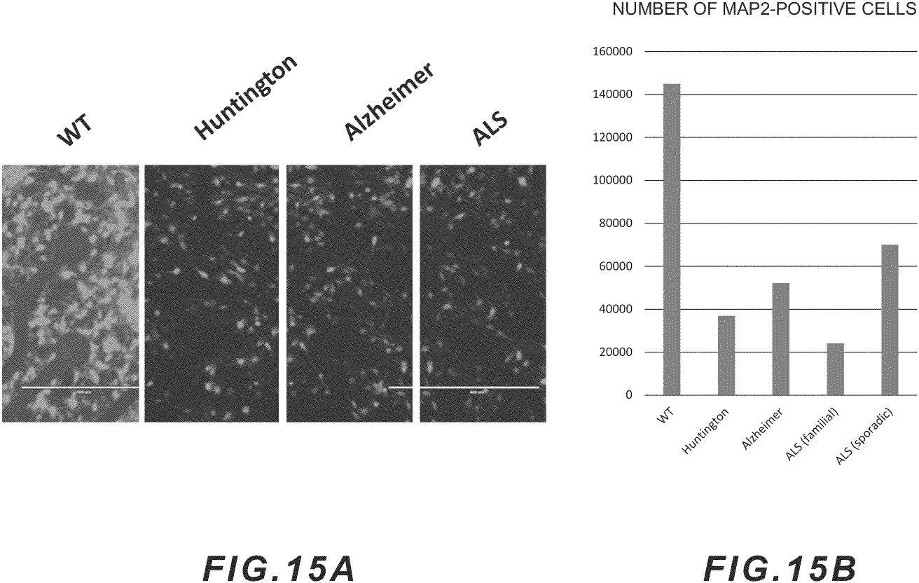

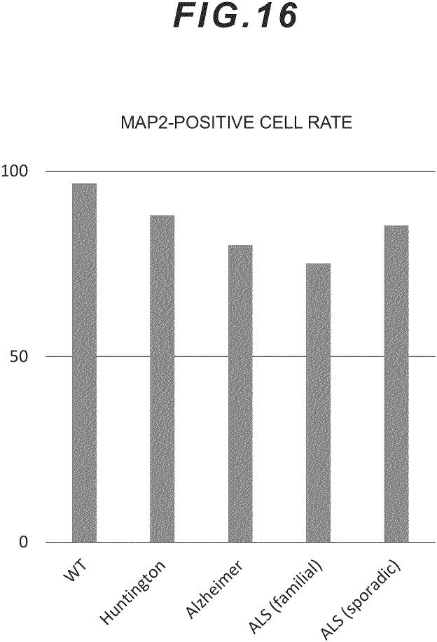

As shown in FIG. 14, the number of the nerve cells differentiated from the iPS cells derived from the neurological disease patients was smaller than the number of the nerve cells differentiated from the iPS cells derived from the healthy subjects (WT) . When nerve cells on the 21^{st} day differentiated in the same method as in Example 1 except that mouse monoclonal antibodies against MAP2 were used were stained, as shown in FIGS. 15A and 15B, the positive rate of MAP2 in the nerve cells differentiated from the iPS cells derived from the neurological disease patients was lower than the positive rate of MAP2 in the nerve cells differentiated from the iPS cells derived from the healthy subjects. In addition, as shown in FIG. 16, 21 days after the differentiation factors were introduced into the iPS cells, the proportion of MAP2-positive cells in TUJ1-positive cells differentiated from the iPS cells derived from the neurological disease patients was lower than the proportion of MAP2-positive cells in TUJ1-positive cells differentiated from the iPS cells derived from the healthy subjects, and the degree of maturity of nerve cells was low.

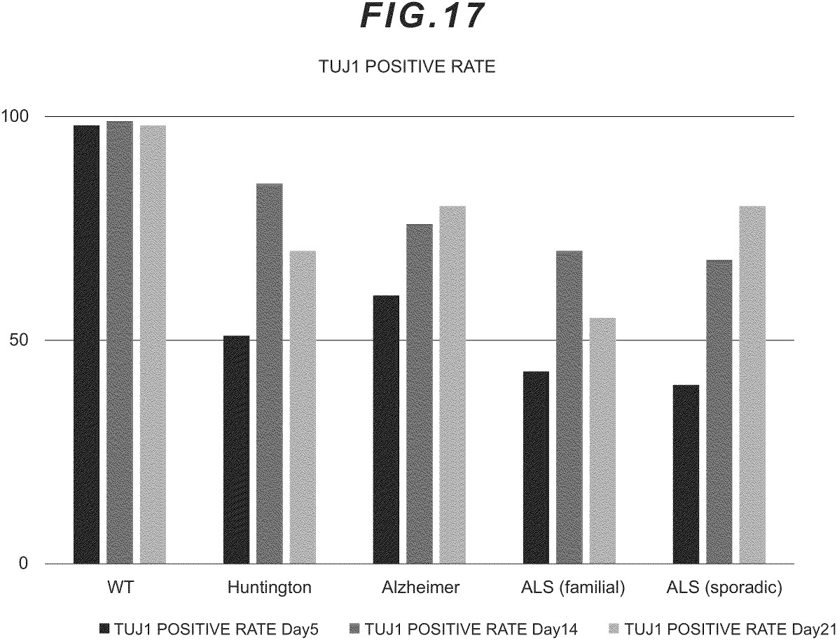

When the nerve cells were stained using mouse monoclonal antibodies against TUJ1, as shown in FIG. 17, the positive rate of TUJ1 in the nerve cells differentiated from the iPS cells derived from the neurological disease patients was lower than the positive rate of TUJ1 in the nerve cells differentiated from the iPS cells derived from the healthy subjects.

### Example 7: Length of Axon

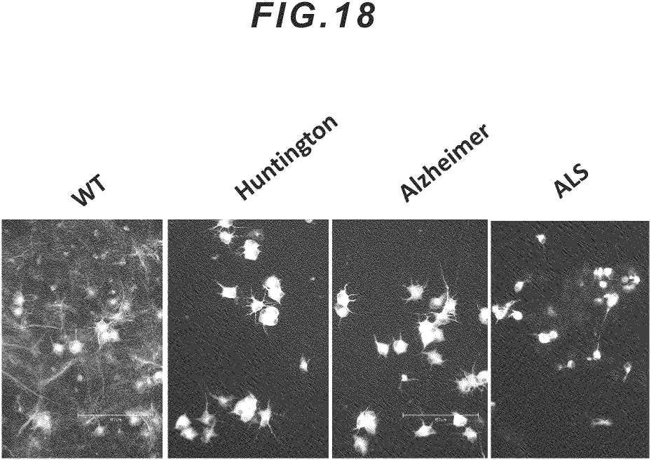

In the same manner as in Example 6, iPS cells derived from healthy subjects, iPS cells derived from amyotrophic lateral sclerosis (ALS) patients, iPS cells derived from Huntington’s disease patients, and iPS cells derived from Alzheimer’s disease patients were prepared, and the iPS cells were differentiated into nerve cells. 21 days after the differentiation factors were introduced into the iPS cells, mouse monoclonal antibodies against MAP2 were used to stain the differentiated nerve cells. As a result, as shown in FIG. 18, the axons of the nerve cells differentiated from the iPS cells derived from the neurological disease patients were shorter than the axons of the nerve cells differentiated from the iPS cells derived from the healthy subjects.

### Example 8: Action Potential

In the same manner as in Example 6, iPS cells derived from healthy subjects, iPS cells derived from amyotrophic lateral sclerosis (ALS) patients, iPS cells derived from Huntington’s disease patients, and iPS cells derived from Alzheimer’s disease patients were prepared, and the iPS cells were differentiated into nerve cells.

A multi-electrode array system (MEA system, commercially available from Axion) that can measure an action potential of nerve cells was prepared. 2 days after the differentiation factors were introduced into the iPS cells, glial cells were seeded in a plate (MEA plate, commercially available from Axion), and the glial cells were cultured in the plate. 4 days after the differentiation factors were introduced into the iPS cells, the differentiated nerve cells were seeded on the glial cells. Then, the nerve cells were cultured while replacing the medium with an N2B27 medium once every 5 days. 42 days after the differentiation factor were introduced into the iPS cells, the action potential of the differentiated nerve cells was measured using a multi-electrode array system.

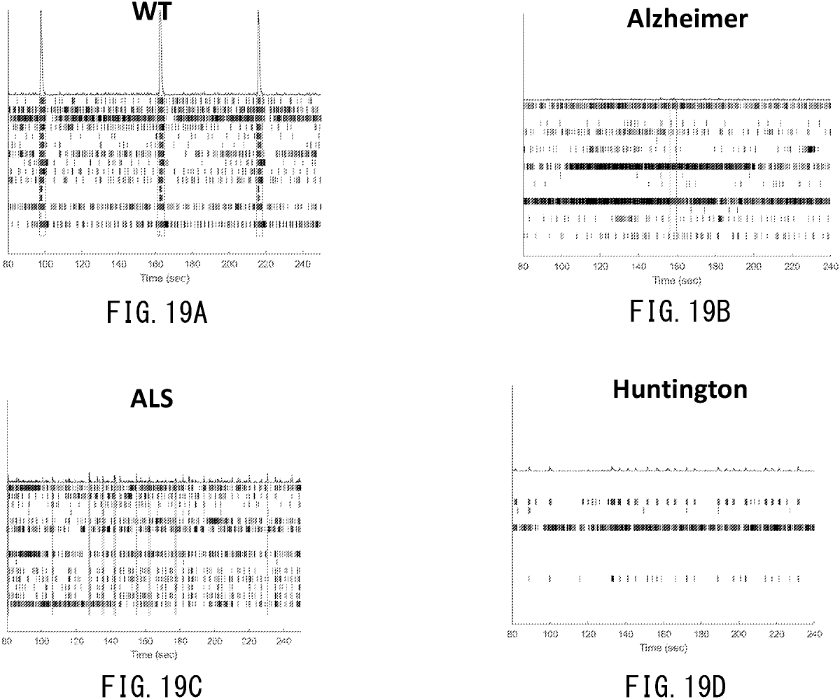

As a result, as shown in FIGS. 19A to 19D, in the nerve cells differentiated from the iPS cells derived from the healthy subjects, action potentials were high and the action potentials coordinated and synchronized via synapses were observed. On the other hand, in the nerve cells differentiated from the iPS cells derived from the neurological disease patients, action potentials were low, and action potentials coordinated and synchronized via synapses were not observed.

### Example 9: Expression of Protein

In the same manner as in Example 6, iPS cells derived from healthy subjects, and iPS cells derived from two amyotrophic lateral sclerosis (ALS) patients were prepared, and the iPS cells were differentiated into motor neurons.

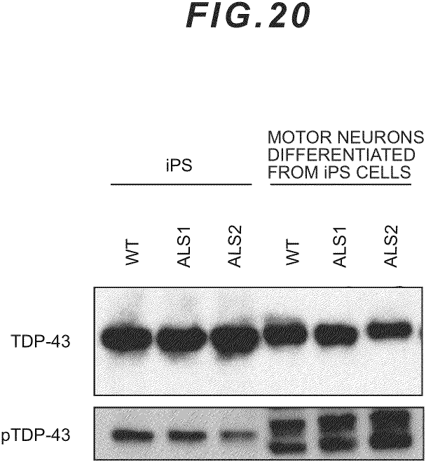

When proteins were extracted from the differentiated nerve cells and Western blotting was performed, as shown in FIG. 20, TDP-43 was more strongly phosphorylated in the nerve cells differentiated from the iPS cells derived from the ALS patients (ALS1 and ALS2) than the in nerve cells differentiated from the iPS cells derived from the healthy subjects.

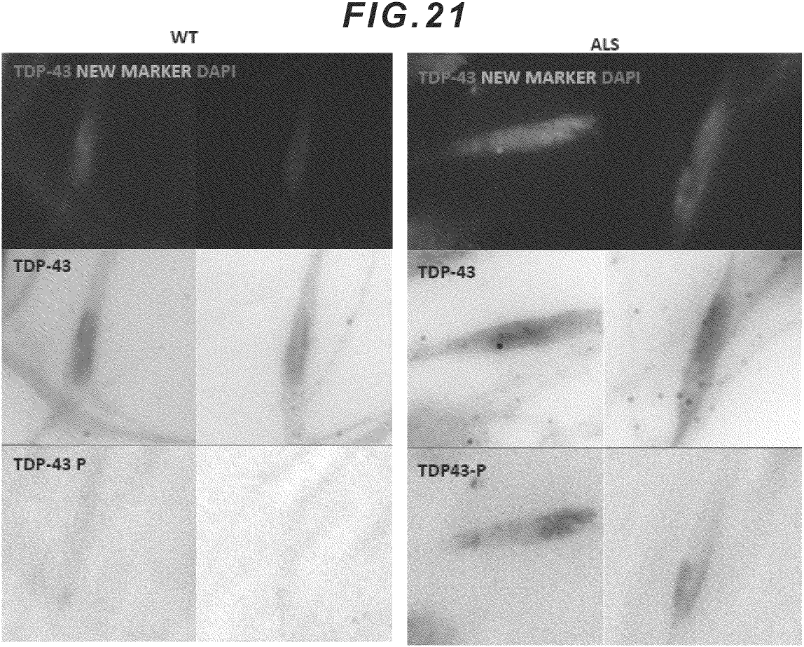

14 days after the differentiation factors were introduced into the iPS cells, the differentiated nerve cells were stained using mouse monoclonal antibodies against TDP43 and mouse monoclonal antibodies against phosphorylated TDP43. As a result, as shown in FIG. 21, compared to the nerve cells differentiated from the iPS cells derived from the healthy subjects, in the nerve cells differentiated from the iPS cells derived from the ALS patients, phosphorylated TDP-43 was more strongly expressed and localized in the cytoplasm.

### Example 10: Expression of Protein

In the same manner as in Example 6, iPS cells derived from healthy subjects, and iPS cells derived from amyotrophic lateral sclerosis (ALS) patients were prepared, and the iPS cells were differentiated into nerve cells.

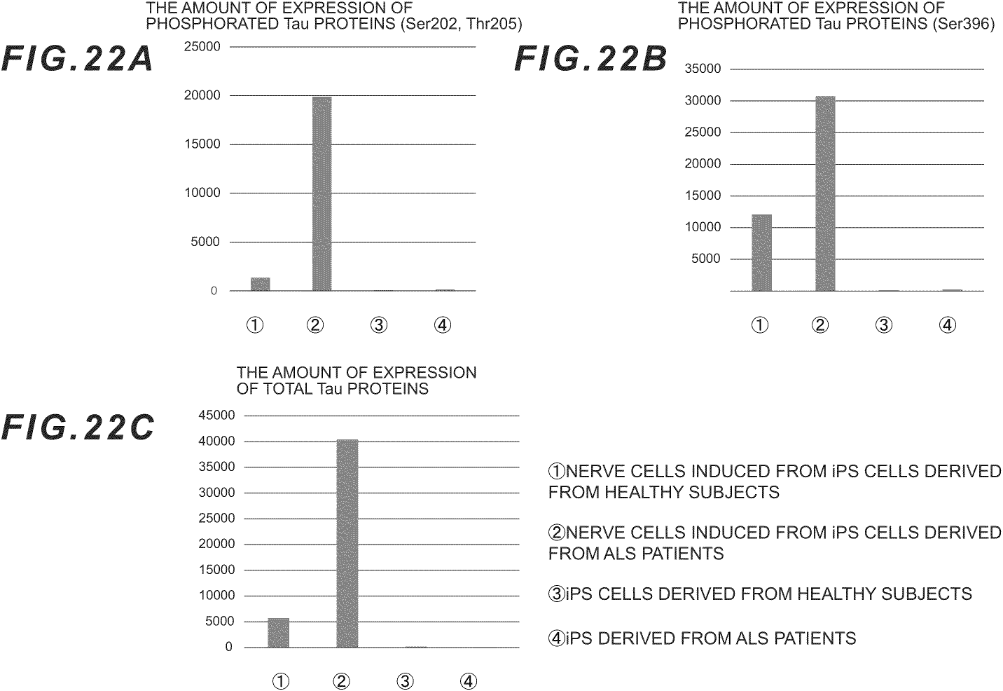

21 days after the differentiation factors were introduced into the iPS cells, when proteins were extracted from the differentiated nerve cells and Western blotting was performed, as shown in FIGS. 22A to 22C, compared to the nerve cells differentiated from the iPS cells derived from the healthy subjects, in the nerve cells differentiated from the iPS cells derived from the ALS patients, the amount of expression of Tau proteins and phosphorylated Tau proteins increased.

What is claimed is:

1. A method for producing a nerve cell, comprising introducing NGN1 or NeuroD4 as a differentiation factor into a stem cell.

2. The method for producing the nerve cell according to claim 1, further comprising introducing MYT1L as a differentiation factor into the stem cell.

3. The method for producing the nerve cell according to claim 1,
wherein the differentiation factor is RNA.

4. A method for producing a motor neuron, comprising introducing NGN2, ISL2, and LHX4 as differentiation factors into a stem cell.

5. The method for producing the motor neuron according to claim 4,
wherein the NGN2, ISL2, and LHX4 RNAs are introduced into the stem cell.

6. A method for producing a motor neuron, comprising introducing NGN1, ISL2, and LHX4 as differentiation factors into a stem cell.

7. The method for producing the motor neuron according to claim 6,
wherein the NGN1, ISL2, and LHX4 RNAs are introduced into the stem cell.

8. A method for producing a motor neuron, comprising introducing NeuroD4, ISL2, and LHX4 as differentiation factors into a stem cell.

9. The method for producing a motor neuron according to claim 8,
wherein the NeuroD4, ISL2, and LHX4 RNAs are introduced into the stem cell.

10. A method for screening a neurological disease therapeutic drug, comprising:
introducing a differentiation factor into a pluripotent stem cell derived from a neurological disease patient and differentiating the cell into a neural cell;
applying a candidate drug to a cell during differentiation of the pluripotent stem cell into the neural cell or to the differentiated neural cell; and
screening the candidate drug on the basis of the neural cell.

11. The method for screening the neurological disease therapeutic drug according to claim 10,
wherein the candidate drug is screened based on the differentiation efficiency of the neural cell.

12. The method for screening the neurological disease therapeutic drug according to claim 10,
wherein the candidate drug is screened based on the survival rate of the neural cell or the number of the neural cell.

13. The method for screening the neurological disease therapeutic drug according to claims 10,
wherein the candidate drug is screened based on the length of an axon of the neural cell.

14. The method for screening the neurological disease therapeutic drug according to claim 10,
wherein the candidate drug is screened based on an action potential of the neural cell.

15. The method for screening the neurological disease therapeutic drug according to claim 10,
wherein the candidate drug is screened based on transmission of the action potential between the neural cells.

16. The method for screening the neurological disease therapeutic drug according to claim 10,
wherein the candidate drug is screened based on expression of a protein in the neural cell.

17. The method for screening the neurological disease therapeutic drug according to claim 10,
wherein the candidate drug is screened based on a change in localization of a protein in the neural cell.

18. The method for screening the neurological disease therapeutic drug according to claim 10,
wherein the candidate drug is screened based on phosphorylation of a protein in the neural cell.

19. The method for screening the neurological disease therapeutic drug according to claim 16,
wherein the protein is a nuclear localized protein.

20. The method for screening the neurological disease therapeutic drug according to claim 10, further comprising
removing an undifferentiated cell.
